(19) United States
(12) Patent Application Publication
(10) Pub. No.: US 2004/0073277 A1
(43) Pub. Date: Apr. 15, 2004

(54) High fluence rate activation of photosensitizers for dermatological applications

(76) Inventors: Roy G. Geronemus, New York, NY (US); Macrene Renee Alexiades-Armenakas, New York, NY (US); Kathleen McMillan, Concord, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/407,921

(22) Filed: Apr. 4, 2003

Related U.S. Application Data
(60) Provisional application No. 60/370,253, filed on Apr. 5, 2002.

(51) Int. Cl. A61N 1/00

(52) U.S. Cl. 607/88

(57) ABSTRACT

Treatment of neoplastic or non-neoplastic dermatological conditions is achieved by administration and photodynamic activation of photosensitizers and pro-photosensitizers by coherent and/or incoherent light. The treatment does not cause clinically signification side effects such as purpura of the treated skin.

### FIELD OF THE INVENTION

This invention relates generally to the field of dermatology, and more particularly to a method of treating certain dermatological conditions by photodynamic activation of photosensitizers in the skin region affected by the conditions.

### BACKGROUND OF INVENTION

5-Aminolevulinic acid (ALA) and related compounds have been employed for the treatment of numerous neoplastic and non-neoplastic dermatologic conditions. ALA when administered topically localizes in basal cell carcinoma, actinic keratoses, viral warts, and other lesional skin tissue. ALA is also taken up by the epidermis and skin appendages (hair follicles and sebaceous glands) of normal tissue. After uptake by lesional or normal skin tissues, ALA is metabolized to produce protoporphyrin IX (PpIX), a photodynamic sensitizer that absorbs light in the visible region of the electromagnetic spectrum. Consequently, application of ALA or its derivatives followed by irradiation with the appropriate dosage of visible light leads to the photodynamic reactions that result in therapeutic injury to skin tissue.

ALA photodynamic therapy (ALA-PDT) in dermatologic applications has employed light sources that operate in a continuous mode. PpIX has an absorption band located in the blue spectral region (410 nm Soret band) and four weaker absorption bands in the 500 to 650 nm visible region (Peng et al. (1997) 5-Aminolevulinic Acid-Based Photodynamic Therapy CANCER 79: 2282-2308). Thus, light sources operating from the blue to red wavelength range may be used for ALA-PDT. Continuous wave (cw) lasers including cw dye and diode lasers have been used in clinical research for ALA-PDT. For dermatologic application where optical fiber delivery of light to the irradiation site is unnecessary, less costly conventional non-laser incoherent light sources can produce equivalent clinical results. Conventional incoherent light sources including incandescent lamps, xenon arc lamps, metal halide lamps, fluorescent tubes, and light emitting diodes have been used for PDT. These cw lasers and non-laser light sources are used at fluence rates of 10 to 500 milliwatts per square centimeter (mWcm⁻²) (Morton et al. (2002) Guidelines For Topical Photodynamic Therapy: Report Of A Workshop Of The British Photodermatology Group BR. J. DERMATOL. 146: 552-567).

At the irradiances typically used, several minutes or more are required to deliver the total dosage of light for optimal activation of the photosensitizer. For example, in the treatment of actinic keratoses (solar keratoses), basal cell carcinoma (BCC), and Bowen's disease using a lamp with peak output at about 630 nm and fluence rate 105-168 mWcm⁻², the total dosage was 105 J cm⁻² (Varma et al. (2001) Bowen 's Disease, Solar Keratoses And Superficial Basal Cell Carcinomas Treated By Photodynamic Therapy Using A Large-Field Incoherent Light Source BR. J. DERMATOL. 144: 567-574). Soler et al used a filtered halogen lamp with output between 550 and 700 nm and fluence rate 150 to 200 mWcm⁻² for a total dosage of 100 J cm⁻² to treat BCC (Solar et al. (1999) Photodynamic Therapy By Topical Aminolevulinic Acid, Dimethylsulphoxide And Curettage In Nodular Basal Cell Carcinoma: A One-Year Follow-Up Study ACTA. DERM. VENEREOL. 79: 204-206). Morton et al. reported using a lamp with output 630±15 nm and irradiance 20 to 86 mWcm⁻² to deliver a total dosage of 100 J cm⁻² in the treatment of BCC (Morton et al. (2001) Photodynamic Therapy For Large Or Multiple Patches Of Bowen Disease And Basal Cell Carcinoma ARCH DERMATOL. 137: 319-324). For BCC of the eyelid, Wang and coworkers used a dye laser tuned to 635 to deliver a total dose of 60 J cm⁻² at a fluence rate below 110 mWcm⁻² (Wang et al. (1999) Photodynamic Therapy Utilizing Topical δ-Aminolevulinic Acid In Non-Melanoma Skin Malignancies Of The Eyelid And The Periocular Skin ACTA OPHTHALMOL. SCAND. 77: 182-188). In general, for BCC, red light is used to maximize the effective depth of treatment, and the total light dose is in the range of 54 to 540 J cm⁻² (Morton et al. (2002), supra).

In ALA-PDT for treatment of actinic keratoses, both blue and red light has been used. Calzavara-Pinton and coworkers used a 630 nm cw dye laser to treat actinic keratoses with a total dose of 60 to 80 cm⁻² and fluence rate 100 mWcm⁻² (Calzavara-Pinton (1995) Repetitive Photodynamic Therapy With Topical Delta-Aminolevulinic Acid As An Appropriate Approach To The Routine Treatment Of Superficial Non-Melanoma Skin Tumors J PHOTOCHEM. PHOTOBIOL.

B 29: 53-7). Although blue light does not penetrate as deeply through tissue as red light, actinic keratoses may be effectively treated with blue light because of the superficial nature of these lesions. A total light dosage of 10 J cm⁻² delivered at a fluence rate of 10 mWcm⁻² at 417±5 nm has been used for treatment of actinic keratoses (Morton et al. (2002), supra). The lower total light dosage is effective only because the Soret absorption band of PpIX is much stronger than the longer wavelength absorption bands located between 500 and 650 nm.

Side effects of ALA-PDT as using either conventional light sources or cw lasers in either the red or blue spectral regions as described above include pain, itching, edema, erythema, erosion, crusting, and dyschromia. Pain may limit the dosage of light that can be given to the patient, potentially reducing treatment efficacy. Although the long-term cosmetic results of ALA-PDT are typically favorable, the side effects occurring with healing in the days and weeks after treatment are for the patient a negative consequence of treatment. Also treatment and recovery times were typically long.

The extent and significance of the side effects of ALA-PDT has been described in the literature. Hongcharu and co-workers reported applying 20% ALA for 3 hours to skin on the back in subjects with acne, followed by irradiation with a broadband lamp (550 to 700 nm) at a total dosage of 150 J/cm² (Hongcharu et al. (2000) Topical ALA-Pholodynamic Therapy For The Treatment Of Acne Vulgaris J. INVEST. DERMATOL. 115:183-192). Acne improved, but side effects included erythema, edema, exfoliation, and long-lasting hyperpigmentation. Itoh and coworkers treated acne patients with ALA and a much lower light dose (13 J/cm²) using red light from a halogen lamp, and reported an excellent therapeutic response although still accompanied by significant side effects including edematous erythema, epidermal exfoliation and irritation lasting several days, and pigmentation changes (Itoh et al. (2001) Photodynamic Therapy Of Acne Vulgaris With Topical Delta-Aminolaevulinic Acid And Incoherent Light In Japanese Patients BR. J. DERMATOL. 144: 575-579). These authors noted that skin treated required one month to return to normal.

Similar observations of acute side effects have been reported when ALA-PDT is used for treatment of neoplastic skin conditions. Recently, Markham and Collins described treatment of actinic keratoses with ALA followed 4 hours later by irradiation with 580 to 740 nm light from a lamp, for a total light dosage of 20 J/cm². Even at this relatively low light dose, erythema and skin erosions occurred. Accordingly, Markham and Collins reported that “the main drawback of topical 5-ALA PDT is its acute adverse effects such as discomfort and erosions for 7 days post treatment” (Markham et al. (2001) Topical 5-Aminolaevulinc Acid Photodynamic Therapy For Extensive Scalp Actinic Keratoses BR. J. DERMATOL. 145: 502-504).

In addition to the applications mentioned above, where ALA-PDT has been used to treat lesional skin, both neoplastic and non-neoplastic, there are conditions of normal skin which may be amenable to the methodology. One example is excessive or unwanted hair which is reduced as a result of photodynamic injury to hair follicles (Grossman et al. (1995) PDT For Hhirsutism LASERS SURG. MED. 7(Supp.): 44). Another is photoaged or wrinkled skin, which has been reported to improve following ALA-PDT (Ruiz-Rodriguez et al. (2002) Photodynamic Photorejuvenation DERMATOL. SURG. 28: 742-744). In these cosmetic applications, the acute side effects of ALA-PDT are particularly disadvantageous

Karrer et al. have described the use of a pulsed dye laser for ALA-PDT (Karrer et al. (1999) Long-Pulse Dye Laser For Photodynamic Therapy—Investigations In Vitro And In Vivo LASERS SURG. MED. 25: 51-9). Actinic keratoses were treated with either the long pulse dye laser or a conventional lamp after application of 20% ALA for 6 hours to the area of the lesion. The dye laser operated at a wavelength of 585 nm, near a relative maximum in the PpIX spectrum. The pulse duration was 1.5 milliseconds, and the total light dosage was 18 J/cm². Efficacy of pulsed dye laser and conventional lamp were similar. Pain was significantly lower when the pulsed dye laser was used, but other side effects including erythema and crusting that lasted for up to two weeks after treatment were similar for the two light sources. Unlike the conventional lamp, the pulsed dye laser also produced purpura, a dark discoloration of the skin, which lasted for up to two weeks after treatment. Thus, the post-operative morbidity and cosmesis of ALA-PDT was not improved by the pulsed dye laser.

### SUMMARY OF THE INVENTION

The present invention provides a method for treating various dermatologic conditions with minimal-to-absent side effects such as purpura of the treated skin. The method of the invention is based on the surprising discovery that a high fluence rate can be used for effective treatment while the overall fluence is kept below the purpura threshold.

In one aspect, the invention is generally directed to a method for treating a neoplastic or non-neoplastic dermatologic condition. In one embodiment, a photosensitizer or a pro-photosensitizer is administered to a section of the skin affected with a neoplastic or non-neoplastic condition. The photosensitizer or pro-photosensitizer is allowed to accumulate in the skin tissue affected with the neoplastic or non-neoplastic condition. The section of the skin affected by the neoplastic or non-neoplastic condition is irradiated with a beam of light that has a wavelength between about 500 nm and about 650 nm, a fluence rate between about 100 W/cm² and about 40 MW/cm², and a fluence of less than about 60 J/cm². The irradiation causes a therapeutic injury to the section of skin affected by the neoplastic or non-neoplastic without causing purpura of the skin, i.e., the treated skin region is substantially free or completely free of purpura. Treatment parameters are selected such that no clinically significant purpura results from the treatment. The purpura threshold, the fluence rate, and the fluence may be statistically estimated and empirically determined and selected according to the characteristics of the individual patient.

In another aspect, the invention is generally directed to a method for treating a neoplastic or non-neoplastic dermatologic condition. In one embodiment, a pro-photosensitizer is administered to a section of the skin affected with a neoplastic or non-neoplastic condition. The pro-photosensitizer is allowed to accumulate and metabolize in the skin tissue affected with the neoplastic or non-neoplastic condition. The section of the affected skin is irradiated with a pulsed laser beam that has a wavelength between about 500 nm and 650 nm, a fluence less than about 20 J/cm², and a pulse duration between about 10 microseconds and 40 milliseconds. The irradiation causes a therapeutic injury to the section of skin affected by the neoplastic or non-neoplastic condition without causing purpura of the skin.

### BRIEF DESCRIPTION OF THE DRAWINGS

The objects and features of the invention may be more fully understood by reference to the following drawings, in which:

FIG. 1 is a graph showing the absorption bands of PpIX produced as a result of ALA metabolism.

FIG. 2 is a graph showing the visible absorption spectra of oxyhemoglobin (solid line) and deoxyhemoglobin (dotted line).

### DESCRIPTION OF INVENTION

The present invention is generally directed to a method for activation of photodynamic sensitizers in dermatologic application. This invention is based on the finding that ALA-PDT using a pulsed light source has efficacy equivalent to conventional ALA-PDT but with fewer and minimal side effects, when the light source is operated in a manner not appreciated by the prior art.

Specifically, in one embodiment of the invention, a pulsed dye laser operating at 595 nm and with pulse duration in the 3 milliseconds to 40 milliseconds range was found to activate PpIX following ALA application to lesional skin, such that the lesion was successfully treated and without the side effects and morbidity associated with standard ALA-PDT. The pulsed dye laser was operated below the purpura threshold of the skin at a fluence of about 3.0 to about 7.5 J/cm². The examples of the findings include treatment of actinic keratoses which occurred without purpura, and with a reduction in pain and in crusting and erosions of the treated lesions commonly associated with ALA-PDT. Treatment was performed and successful with and without surface cooling. Surface cooling provided epidermal protection and additional pain reduction.

These findings were surprising because the total light dosage (fluence) of the pulsed dye laser was considerably lower than that typically used in conventional ALA-PDT using cw lasers or incoherent light sources. Higher light dosages are recommended for effective treatment in ALA-PDT (Morton et al. (2002), supra). Furthermore, the report of Karrer et al. had shown a side effect profile for ALA-PDT using a pulsed dye laser at suprapurpuric fluences that included purpura not seen with conventional ALA-PDT (Karrer et al., supra). The benefit of their use of a pulsed dye laser was limited to some pain reduction and a faster treatment time. Thus, following the publication of Karrer's results in 1999, no further reports of pulsed dye laser for ALA-PDT appeared, prior to the time of the present invention.

The findings that pulsed irradiation can be advantageous for ALA-PDT is also surprising in light of the current understanding of the mechanism of action of photodynamic therapy. Not only are the total light doses as used in the current invention lower than those that have been used to successfully treat skin lesions in the past, but the fluence rates are considerably higher. As used herein, a “fluence rate” is fluence divided by pulse duration. Numerous experimental studies of photodynamic therapy have shown that high fluence rate treatment is less effective than low fluence rate treatment. The reduction in efficacy with higher fluence rates is understood to be a consequence of oxygen depletion (Henderson et al. (2000) Photofrin Photodynamic Therapy Can Significantly Deplete Or Preserve Oxygenation In Human Basal Cell Carcinomas During Treatment, Depending On Fluence Rate CANCER RES. 60: 525-529), and has been observed regardless of the particular photosensitizer being used. For example, it has been shown in a tumor model using the photosensitizer Photofrin that 60 Jcm⁻² delivered at 10 mWcm⁻² had a similar effect as 150 Jcm⁻² at 200 mWcm⁻² or 100 Jcm⁻² at 100 mWcm⁻² (Sitnik et al. (1998) The Effect Of Fluence Rate On Tumor And Normal Tissue Responses To Photodynamic Therapy PHOTOCHEM. PHOTOBIOL. 67: 462-468). Thus, even within the fluence range typically used for ALA-PDT using cw lasers or incoherent light sources (Morton et al., supra), higher fluence rates may reduce efficacy. It may be noted that the fluence rates used in the present invention are even higher. For example, a 10 millisecond (ms) laser pulse with fluence of 7.5 Jcm⁻² corresponds to a fluence rate of 750 Wcm⁻². This fluence rate is more than 3 orders of magnitude greater than the fluence rates used in conventional ALA-PDT. However, this fluence rate is well below the peak fluence rate value of 4×10⁴ Wcm⁻² which is the estimated threshold for a reduction in PDT efficacy due to saturation (Sterenborg et al. (1996) Photodynamic therapy with pulsed light sources: at theoretical analysis PHYS. MED. BIOL. 41: 835-49).

Based on the current understanding of the mechanisms of photodynamic therapy, the low light dosage and the very high fluence rate used in the present invention would both be expected to limit the efficacy of treatment. However, according to the present invention the use of a laser at relatively low light dosage and high fluence rate is not only effective but advantageous.

In one aspect, the invention is generally directed to a method for treating a neoplastic or non-neoplastic dermatologic condition. In one embodiment, a photosensitizer or a pro-photosensitizer is administered to a section of the skin affected with a neoplastic or non-neoplastic condition. The photosensitizer or pro-photosensitizer is allowed to accumulate in the skin tissue affected with the neoplastic or non-neoplastic condition. The section of the skin affected by the neoplastic or non-neoplastic condition is irradiated with a beam of light that has a wavelength between about 500 nm and about 650 nm, a fluence rate between about 100 W/cm² and about 40 MW/cm², and a fluence of less than about 60 J/cm². The irradiation causes a therapeutic injury to the section of skin affected by the neoplastic or non-neoplastic without causing purpura of the skin.

As used herein, the term “without causing purpura” means that the treated skin region is substantially free or completely free of purpura. Treatment using the method of the invention could in some cases produce a small amount of darkening of the treated skin region compared to normal skin regions; however, such changes are minimal and clinically insignificant. In other words, the parameters of the treatment are selected such that no clinically significant purpura results from the treatment of the invention. The purpura threshold, the fluence rate, and the fluence is statistically estimated and empirically determined and selected prior to treatment according to the characteristics of the individual patient. Procedures for statistical estimation and empirical testing are known to those skilled in the art. The present invention is also directed to a method of treating normal skin with reduced side effects.

As used herein, the term “photosensitizer” means a photoactive chromophore that can be used in photodynamic therapy. Photosensitizers useful in the practice of the invention are activated by pulsed or scanned coherent or incoherent electromagnetic radiation having a wavelength in the range from about 400 nm to about 800 nm. As will be discussed in more detail below, the parameters of the coherent or incoherent electromagnetic radiation preferably are selected so that the radiation is capable of (i) penetrating the skin to a certain depth, (ii) activating the photosensitizer, and (iii) producing minimal vascular damage clinically evident as purpura.

Photosensitizers useful in the practice of the invention include, for example, chlorins, cyanines, purpurins and porphyrins, for example, benzoporphyrin derivative monoacid (BPD-MA) (available from QLT, Inc., Vancouver, Canada). Other useful photosensitizers include, for example, bacteriochlorins and bacteriopurpurins, such as those described in U.S. Pat. No. 6,376,483 B1, for example 5,10-octaethylbacteriopurpurin, and 5,15-octaethylbacteriopurpurin, or nickel 5,10-bis-acrylate etioporphyrin I. Other useful photosensitizers include xanthenes, for example, rose bengal, or other photosensitizers that may be isolated or derived from natural sources, or synthesized de novo, for example, hypericin (available from Sigma Chemical Co., St. Louis, Mo.). It is understood that this list of photosensitizers including those in Table I is exemplary, and that other photosensitizers currently available or yet to be developed having the appropriate spectral characteristics may also be useful in the practice of the invention.

As used herein, the term “pro-photosensitizer” means any molecule, which when administered to a mammal is capable of being metabolized or otherwise converted to produce a photosensitizer, or is capable of stimulating the synthesis of an endogenous photosensitizer. It is contemplated that the pro-photosensitizer may be converted into a photosensitizer of interest or may stimulate the synthesis of an endogenous photosensitizer at the site of the skin affected with the condition to be treated. Alternatively, the pro-photosensitizer may be converted into a photosensitizer or stimulate the synthesis of an endogenous photosensitizer at a region remote from the target skin region, after which the photosensitizer is transported to the target skin region, for example, via the vasculature. When a pro-photosensitizer is used, the pro-photosensitizer is allowed to accumulate, metabolize, covert, or otherwise stimulate the synthesis of a photoactive chromophore.

It is contemplated that pro-photosensitizers useful in the practice of the invention include, for example, precursors of PpIX, for example, ALA (available from Sigma Chemical Co., St. Louis, Mo.), ALA derivatives, such as, ALA esters (e.g., ALA-methyl ester, ALA-n-pentyl ester, ALA-n-octyl ester, R,S-ALA-2-(hydroxymethyl)tetrahydropyranyl ester, N-acetyl-ALA, and N-acetyl-ALA-ethyl ester). See, e.g., U.S. Pat. No. 6,034,267.

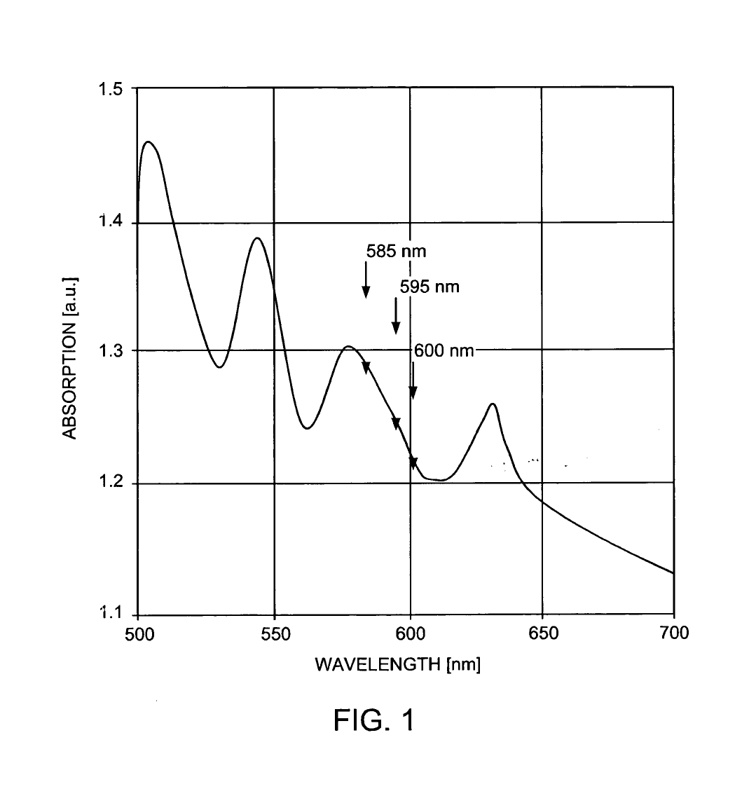

FIG. 1 is a graph showing the absorption bands of PpIX, which is produced as a result of ALA metabolism in a mammal. PpIX has an absorption band located in the blue spectral region (the 410 nm Soret band) and other weaker absorption bands in the 500 to 650 nanometer visible region, namely at about 506 nm, 546 nm, 578 nm, and 635 nm.

Table 1 lists several photosensitizers useful in the practice of the invention, and identifies relevant absorption peaks for each of the listed photosensitizers.

TABLE 1

| | Approximate wavelength of |
|---|---|
| Photosensitizer | selected absorption peaks (nm) |
| chlorin e6 | 410, 658 |
| mono-N-aspartyl chlorin e6 | 664 |
| tin etiopurpurin | 660 |
| lutetium texaphyrin | 470, 730 |
| 5,10-octaethylbacteriopurpurin | 563, 598 |
| 5,15-octaethylbacteriopurpurin | 558, 592 |
| nickel 5, 10-bis-acrylate | 580 |
| etioporphyrin I | |
| protoporphyrin IX | 506, 546, 578, 635 |
| benzoporphyrin derivative mono- | 400, 585, 687 |
| acid | |
| hypericin | 550, 595 |
| rose bengal | 548 |
| hematoporphyrin derivative | 505, 537, 565 |
| Pd(II)-octabutoxyphthalocyanine | 732, 838 |
| Si(IV)-naphthocyanine | 773 |

It is considered that the choice of the appropriate photosensitizer or pro-photosensitizer, formulation, dosage, and mode of administration will vary depending upon several factors including, for example, the skin disorder to be treated, and the age, sex, weight, and size of the mammal to be treated, and may be varied or adjusted according to choice. The photosensitizer or pro-photosensitizer is administered so as to permit an effective amount of photosensitizer to be present in the target region. As used herein, the term “effective amount” means an amount of photosensitizer suitable for photodynamic therapy, i.e., the photosensitizer is present in an amount sufficient to produce a desired photodynamic reaction at the target site. The photosensitizer or prophotosensitizer may be administered in a single dose or multiple doses over a period of time to permit an effective amount of photosensitizer to accumulate in the target region. Fluorescence spectroscopy or other optical detection or imaging techniques may be used to determine whether and how much photosensitizer is present in the target region.

The photosensitizers or pro-photosensitizers may be formulated into delivery systems that deliver or permit the accumulation of photosensitizer to the target tissue. Such formulations may include coupling the photosensitizer or pro-photosensitizer to a specific binding ligand which binds to a target in the tissue of interest, and/or by formulation with a carrier that can deliver the photosensitizer or pro-photosensitizer to the target tissue. It is also contemplated that a combination of one or more photosensitizers and one or more pro-photosensitizers may be used to achieve optimal result of treatment.

In addition, the compositions of the photosensitizer or pro-photosensitizer may be formulated in conventional manner with one or more physiologically acceptable carriers or excipients, according to techniques well known in the art. Compositions may be administered topically, orally or systemically. Under certain circumstances and depending upon the photosensitizer and/or the pro-photosensitizer chosen, topical compositions may be preferred. Topical compositions may include liposomal formulations, emulsions, gels, creams, ointments, sprays, lotions, salves, sticks, soaps, powders, aerosols, drops and any of the other conventional pharmaceutical forms in the art. Ointments and creams may, for example, be formulated with an aqueous or oily base with the addition of suitable thickening and/or gelling agents. Lotions may be formulated with an aqueous or oily base and will, in general, also contain one or more emulsifying, dispersing, suspending, thickening, or coloring agents. Powders may be formed with the aid of any suitable powder base. Drops may be formulated with an aqueous or non-aqueous base also comprising one or more dispersing, solubilizing or suspending agents. Aerosol sprays are conveniently delivered from pressurized packs, with the use of a suitable propellant. In addition, the photosensitizer or pro-photosensitizer may be administered topically using an external energy source, for example, by electrical means (e.g., by iontophoresis) or by ultrasound (e.g., by therapeutic ultrasound).

Alternatively, the photosensitizer or pro-photosensitizer may be provided in a form adapted for oral, or parenteral administration, for example, by intramuscular, intradermal, subcutaneous, intraperitoneal, or intravenous injection. Alternative pharmaceutical forms thus include plain or coated tablets, capsules, suspensions, and solutions containing the active component optionally together with one or more inert conventional carriers and/or diluents, for example, with corn starch, lactose, sucrose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, stearylalcohol, carboxymethylcellulose or fatty substances such as hard fat or suitable mixtures thereof.

In one embodiment, the pro-photosensitizer is ALA. This pro-photosensitizer is well established in the photodynamic treatment of neoplastic and non-neoplastic skin lesions. ALA is a precursor in the synthesis of PpIX, a naturally occurring photosensitizer, which itself is a precursor in the synthesis of heme. See, e.g., U.S. Pat. Nos. 5,079,262, 5,211,938, and 5,955,490. Both ALA and PpIX are naturally present in the body and, therefore, in general, induce few or no side-effects. It is believed that all nucleated cells have at least a minimal capacity to synthesize PpIX. Typically, the synthesis of PpIX is regulated so that it is produced in cells at a rate just sufficient to satisfy the need for heme. Although the synthesis of ALA is a rate-limiting step in the synthesis of heme, it is believed that this step can be bypassed by providing exogenous ALA, or other precursors of PpIX.

ALA is an effective inducer of PpIX when given orally, topically, or by injection. See, U.S. Pat. Nos. 5,079,262, 5,211,938, and 5,955,490. The oral and parenteral routes lead to the induction of clinically useful concentrations of PpIX in the skin. Furthermore, because PpIX apparently can be synthesized in the skin, ALA may be applied topically to the target region. For dermatological applications, ALA usually is administered topically.

It is contemplated that ALA may be applied topically as an ointment containing from about 1% to about 40%, more preferably from about 5% to about 30%, and most preferably from about 10% to about 20% (wt/wt) of ALA in a suitable pharmaceutically acceptable carrier or excipient. The typical formulation may comprise a solution, emulsion, cream, or liposomal formulation. Furthermore, ALA may be delivered iontophoretically to the surface of the skin (Rhodes et al. (1997) J. INVEST. DERMATOL. 108: 87-91.). Alternatively, ALA may be administered orally in solution, for example, fruit juice, at a final dosage of about 1 to about 60 mg/kg of body weight, at a dosage of about 5 to about 40 mg/kg of body weight, or at a dosage of about 10 to about 20 mg/kg of body weight.

It should be noted that the photosensitizer or pro-photosensitizer dosage should be adjusted with respect to the irradiation parameters, including, for example, wavelength, fluence, fluence rate, irradiance, duration of the light, and the time interval between administration of the photosensitizer or pro-photosensitizer and the irradiation, and the cooling parameters, if surface cooling is desired. All of these parameters should be adjusted to produce a photodynamic reaction resulting from activation of the photosensitizer in the target region that is effective with minimal side effects.

Appropriately selected coherent or incoherent radiation is capable of penetrating the skin and activating the photosensitizer present in the region of irradiation. The wavelength of the coherent or incoherent radiation is important, as it has been shown that between 1 and 10 percent of incident red light (600-700 nm) can pass through a slab of human tissue 1 cm thick, whereas only 0.001 percent or less of blue light (about 400 nm) can pass through the same thickness of human tissue (U.S. Pat. No. 5,079,262). By way of example, Table 2 lists the estimated skin penetration depth of light of differing wavelengths.

TABLE 2

| | Esimated penetration depth |
|---|---|
| Wavelength (nm) | (mm)¹ |
| 440 | 0.6 |
| 460 | 1.0 |
| 480 | 1.3 |
| 500 | 1.6 |
| 520 | 1.7 |
| 540 | 1.2 |
| 560 | 1.7 |
| 580 | 1.4 |
| 600 | 4.2 |
| 620 | 5.2 |
| 640 | 5.8 |
| 660 | 6.3 |

The penetration depths were estimated by the method of Jacques (Jacques (1992) PROC. SPIE. 1645: 155-165) assuming skin with 1% volume fraction blood.

Suitable light sources useful in the practice of the invention include (i) incoherent light sources optionally with one or more light filters, and (ii) coherent light sources. Suitable incoherent light sources include, for example, flash lamps, and filtered flash lamps. Suitable coherent light sources include, for example, pulsed lasers (e.g., pulsed diode lasers such as gallium arsenide diode lasers) and flashlamp pumped pulsed dye lasers (e.g., the 585 nm pulsed dye laser (C Beam from Candela Corp., Wayland, Mass.), and the 595 nm pulsed dye laser (V Beam from Candela Corp., Wayland, Mass.). Other suitable pulsed lasers include pulsed solid state lasers, for example flashlamp pumped alexandrite lasers and neodymium:YAG lasers. Other suitable coherent light sources include cw lasers that are scanned over the treatment area, for example, scanned cw dye lasers, frequency doubled neodymium:YAG lasers, or cw diode lasers.

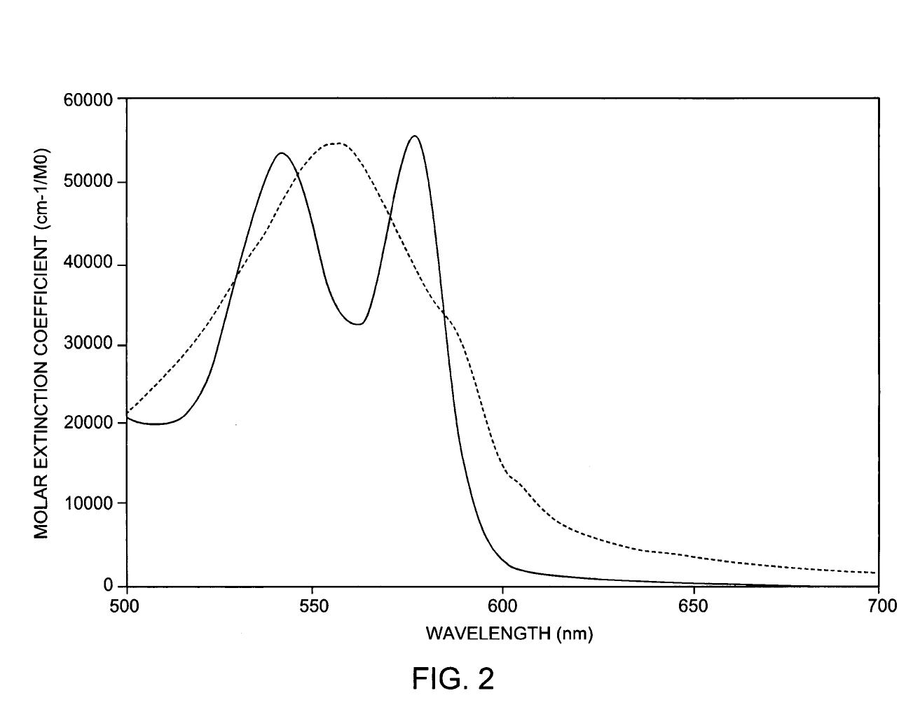

The method of the invention involves photodynamic therapy with reduced side effects, including specifically an absence of clinically significant purpura. Purpura is indicative of vascular coagulation and extravasation, and typically results from a localized and transient temperature in the range of about 60° C. to about 100° C. Heating of blood vessels to these temperatures can be produced using light sources, when sufficient light energy is absorbed by blood or blood components. FIG. 2 shows the visible absorption spectra of oxyhemoglobin (solid line) and deoxyhemoglobin (dotted line), which are the major light absorbing components of blood. Hemoglobin has absorption peaks at about 542 nm and about 576 nm. Deoxyhemoglobulin has a broad absorption peak with maximal absorption at about 556 nM. Some photosensitizer absorption peaks, including those of PpIX, overlap with the absorption bands of hemoglobin and deoxyhemoglobin in the visible region (see Table I).

It is understood that when using a light source having a wavelength that is absorbed by both the photosensitizer and blood or blood components, the irradiation parameters (pulse duration and total light dose) is adjusted so that irradiation produces minimal clinically observable purpura. The required adjustment can be predicted from model calculations of the interaction of light with tissue (see, e.g., De Boer et al. (1996) Thermolysis Of Port-Wine-Stain Blood Vessels. Diameter Of A Damaged Blood Vessel Depends On The Laser Pulse Length LASERS MED. SCI. 11: 177-180; Kimel et al. (2002) Vascular Response To Laser Photothermolysis As A Function Of Pulse Duration, Vessel Type, And Diameter. Implications For Port Wine Stain Laser Therapy LASER SURG. MED. 30: 160-169; Van et al. (1997) Wavelengths For Port Wine Stain Laser Treatment. Influence Of Vessel Radius And Skin Anatomy PHYS. MED. BIOL. 42: 41-50; Nelson et al. (1995) Laser Pulse Duration Must Match The Estimated Thermal Relaxation Time For Successful Photothermolysis Of Blood Vessels LASERS MED. SCI. 10: 9-12.). Also, for many light sources, the effect of irradiation of skin and/or blood vessels is known from clinical experience. For many light sources, for example commercially available pulsed dye lasers such as the V Beam and C Beam (Candela Corporation), which are designed for the treatment of various skin conditions such as vascular lesions of the skin, medical practitioners are familiar with the range of irradiation parameters that correspond to purpura threshold in skin. Finally, for a particular light source and a particular patient, the precise parameters within this range corresponding to the threshold of purpura production can be determined by irradiated small inconspicuous test sites on the patient, immediately before the light source is used according to the present invention. The purpura threshold has been precisely defined in the literature and is a well-established concept in determining the appropriate total light dose for treatment of vascular and other skin lesions (Garden et al. (1986) Effect Of Dye Laser Pulse Duration On Selective Cutaneous Vascular Injury J. INVEST. DERMATOL. 87: 653-757). However, As noted previously, however, in the present invention it is understood that the total light dose is kept below the purpura threshold to minimize side effects of photodynamic therapy.

It is also contemplated that light sources may be used which correspond to photosensitizer absorption bands located in spectral regions where absorption bands of blood and/or blood components are absent or weak. For example, the absorption band of PpIX located at about 635 nm in the red may be used. A potential advantage of irradiating at wavelengths not well absorbed by blood is that higher total light dosages may be used without inducing purpura. Another potential advantage of irradiating at wavelengths not well absorbed by blood is that the depth of penetration of the light is increased, so that thicker or deeper lesions or tissue may be treated.

The apparatus of the present invention includes a light source that provides a coherent or incoherent light beam with the pre-selected characteristics. The light beam may be a pulsed or scanned laser, flashlamp, or other pulsed incoherent source emitting radiation at wavelengths between about 400 nm and about 800 nm in the visible region of the electromagnetic spectrum, so as to be absorbed by a photosensitizer present in the target skin tissue.

In some embodiments, the coherent or incoherent light has a pulse duration in the range from about 1 microsecond to about 200 milliseconds per pulse, optionally in the range from about 10 microseconds to about 20 milliseconds per pulse, or optionally in the range from about 100 microseconds to about 10 milliseconds per pulse. Pulses with the duration within this range, that consist of a series of shorter micropulses may be used. Scanned lasers or other scanned light sources with exposure times equal to these pulse durations may be used.

In some embodiments, the coherent or incoherent light has a spot size in the range from about 1 millimeter to about 30 millimeters in diameter, or optionally in the range from about 5 millimeters to about 20 millimeters in diameter.

Following administration of the selected photosensitizer or photosensitizer, the area treated is exposed to radiation from the coherent or incoherent light source to achieve the photodynamic effect. The length of time following administration, at which the light exposure takes place will depend on the nature of the composition, and the mode of administration. This length of time can range from about 0.1 to 48 hours post administration, preferably in the range from 0.25 to 25 hours and, more preferably in the range from 0.50 to 15 hours.

The photosensitizer or pro-photosensitizer, for example ALA and related compounds (including the homologues and analogues of ALA) that are metabolized to form PpIX in tissue, are applied to the skin using any of the known methodology, for example in the form of creams, ointments, emulsions, or solutions. The photosensitizer or pro-photosensitizer can also be applied using the techniques of iontophoresis or ultrasound.

In one embodiment, the photosensitizer is PpIX produced by administration of ALA or its derivatives. The apparatus includes a pulsed dye laser operating at between 585 nm and 600 nm, with pulse duration 0.45 to 40 milliseconds. Apparatus with these operational parameters currently exist for treatment of vascular lesions. In another embodiment, the apparatus includes a pulsed dye laser operating at about 610 nm to about 650 nm. In another embodiment, the pulsed laser operates at about 620 nm to about 650 nm. In yet another embodiment, the pulsed laser operates at about 635 nm. The pulsed laser can operate at a wavelength that coincides with other photosensitizer absorption peaks.

The fluence rate is typically within the range of about 100 W/cm² to about 40 MW/cm². In certain embodiments, the fluence rate is within the range of about 300 W/cm² to about 30 MW/cm². In certain embodiments, the fluence rate is within the range of about 500 W/cm² to about 20 MW/cm². In certain embodiments, the fluence rate is within the range of about 500 W/cm² to about 10 MW/cm². For example, a commercially available 595 nm pulsed dye laser (V Beam from Candela Corporation) can be set to achieve a fluence rate of about 20 MW/cm² at a fluence of about 10 J/cm² and about 10 milliseconds (comprised of multiple short pulses).

Another commercially available 585 nm pulsed dye laser (C Beam from Candela Corporation) can be set to achieve a fluence rate of about 11 MW/cm² at a fluence of about 5.0 J/cm² and about 0.5 milliseconds (comprised of multiple short pulses). A flashlamp may also achieve a desired fluence rate (e.g., in the range from about 500 W/cm² to about 1,000 W/cm²).

The light fluence (e.g., laser light fluence) is adjusted to be less than about 60 J/cm², or below the purpura threshold of skin for any specific combination of wavelength and pulse duration. The spot size on the skin is adjusted to be about 5 to about 10 mm in diameter. In certain embodiments using laser irradiation, the laser fluence is adjusted to be less than about 30 J/cm². In certain embodiments, the laser fluence is adjusted to be less than about 15 J/cm². In certain embodiments, the laser fluence is adjusted to be less than about 12 J/cm². In other embodiments, the laser fluence is adjusted to be less than about 10 J/cm². In certain embodiments, the laser fluence is adjusted to be less than about 7.5 J/cm². In certain embodiments, the laser fluence is adjusted to be less than about 4.0 J/cm². In certain embodiments, the laser fluence is adjusted to be less than about 2.0 J/cm². The spot size may be adjusted to be in the range from about 2 to 20 mm in diameter.

In certain embodiments, the photosensitizer is hypericin, and the apparatus includes a pulsed dye laser operating at between 585 nm and 600 nm, with pulse duration 0.45 to 40 milliseconds. Apparatus with these operational parameters currently exist for treatment of vascular lesions (C Beam, Candela Corporation, Wayland, Mass.). The laser fluence is adjusted to be less than about 20 J/cm², or below the purpura threshold of skin for any specific combination of wavelength and pulse duration. The spot size on the skin is adjusted to be about 5 to about 10 mm in diameter. In certain embodiments using laser irradiation, the laser fluence is adjusted to be less than about 15 J/cm². In certain embodiments, the laser fluence is adjusted to be less than about 12 J/cm². In other embodiments, the laser fluence is adjusted to be less than about 10 J/cm². In certain embodiments, the laser fluence is adjusted to be less than about 7.5 J/cm². In certain embodiments, the laser fluence is adjusted to be less than about 4.0 J/cm². In certain embodiments, the laser fluence is adjusted to be less than about 2.0 J/cm². The spot size may be adjusted to be in the range from about 2 to 20 mm in diameter.

In certain embodiments, the photosensitizer is PpIX produced by administration of ALA or its derivatives. The apparatus includes a filtered flashlamp, operating at a fluence of between 30 and 50 J/cm², using a cut-off filter of 550 or 570 nm. The flashlamp produces a train of 2 or 3 micropulses that are between 1 millisecond and 10 milliseconds in duration, separated by delays of 10 to 60 milliseconds, for a total pulse duration of about 20 milliseconds to about 200 milliseconds. Apparatus with these operational parameters currently exist for treatment of vascular lesions (Vasculight, Lumenis, Israel).

In certain embodiments, the light source, for example an incoherent light source, for example a flashlamp, can be used in combination with a radiofrequency (RF) generator. In certain embodiments, RF can be combined with light from a filtered flashlamp to treat unwanted hair following administration of ALA or its derivatives. RF heating may be combined before, during or after the light pulse, to produce a thermal injury to the hair follicle. In certain cases, this thermal injury combined with the photodynamic injury induced by the photosensitizer and the light pulse may be more effective than the photodynamic injury alone.

In certain embodiments, the neoplastic condition of the skin includes actinic keratosis, skin cancer, Bowen's disease, or dysplasia. In certain other embodiments, the non-neoplastic condition of the skin includes verrucae vulgaris, oily skin, lichen planus, psoriasis or eczema. In yet other embodiments, the non-neoplastic condition of the skin includes one or more forms of acne (e.g., acne vulgaris, acne conglobata, acne comedonica, papularpustular acne, acne inversa, acne fulminans, back acne, acne mechanica), acne rosacea, or sebaceous hyperplasia. In yet other embodiments, the non-neoplastic condition of the skin includes excess or unwanted hair, photodamage, photoaging, or wrinkles.

The treatment results in the reduction of one or more of (i) the surface area of the lesion, (ii) the depth or thickness of the lesion, (iii) the amount and/or coloration, for example, redness, of the lesion, and (iv) the amount of scaling at the site of the lesion. Although the method produces a long lasting effect and, therefore, reduces the number of treatments necessary, it is contemplated that the process may be repeated when desired. In addition, under certain circumstances, the effectiveness of the treatment may be improved by removing keratotic layers from the lesion prior to administration, for example, by topical application, of the photosensitizer or the pro-photosensitizer. Furthermore, under certain circumstances, the effectiveness of treatment may be improved by removing keratotic layers from the lesion after administration, for example, by systemic administration, of the photosensitizer or the pro-photosensitizer, but prior to irradiation. The keratotic layers may be removed by a physical process (e.g., by abrasion), by a chemical process (e.g., by application of a descaling agent, for example, salicylic acid, to the lesion, or by administration of a drug, for example, a topical drug, to the lesion), or by a combination of these are other physical and chemical processes.

Under certain circumstances, it may be advantageous to minimize thermal injury to the epidermis and upper layers of the dermis and/or to reduce pain and discomfort as may arise during the procedure. This may be accomplished by cooling the skin surface prior to, contemporaneous with, and/or after irradiation. Also, it is contemplated that the cooling can be applied at intervals between the pulses of irradiation.

Cooling may be facilitated by one or more cooling systems known and used in the art, e.g., blowing a cold stream of gas or liquid onto the surface of the skin, conductive cooling using a cold contact surface (e.g., U.S. Pat. No. 5,810,801), applying a low boiling point, non-toxic liquid (e.g., tetrafluoroethane or chlorodifluoromethane) onto the surface of the target tissue by evaporative cooling, or a combination of these or other methods of cooling. One example of effective cooling technique is a dynamic cooling device (DCD), such as a DCD manufactured by Candela Corp. (Wayland, Mass.). See, e.g., U.S. Pat. Nos. 5,820,626 and 5,814,040 and PCT/US97/03449.

In light of the foregoing general discussion, the specific examples presented below are illustrative only and are not intended to limit the scope of the invention. Other generic and specific configurations will be apparent to those persons skilled in the art.

### EXAMPLES

### Example 1

This example provides one approach using a topically applied pro-photosensitizer for treating basal actinic keratoses in a human.

Topical 20% 5-aminolevulinic acid (ALA) solution (Levulan, DUSA) is applied to the affected areas. Following a 10 minute-to-18 hour incubation time in a low-light environment, the solution is removed from the skin surface. The area is treated with contiguous, minimally overlapping pulses from a pulsed dye laser operating at 595 nm, with an irradiated spot size of about 10 mm in diameter, pulse duration of about 10 milliseconds, and fluence of about 7.0 J/cm². Dynamic cooling of the epidermis is used during treatment. The procedure may be repeated at monthly intervals if necessary until the lesions are eradicated.

### Example 2

This example provides one approach using a topically applied pro-photosensitizer for treating acne vulgaris and acne scars in a human.

Topical 20% 5-ALA solution (Levulan, DUSA) is applied to the entire affected area on face, torso and extremities for about 45 minutes. The solution is removed from the skin surface. The areas are treated with contiguous, minimally overlapping pulses from a pulsed dye laser operating at 595 nm, with an irradiated spot size of about 7 mm in diameter, pulse duration of about 1.5 milliseconds, and fluence of about 3.0 J/cm². The procedure may be repeated at 2-to-6 week intervals until the acne is cleared.

### Example 3

This example provides one approach using a topically applied pro-photosensitizer for treating acne fulminans in a human.

Topical 20% 5-ALA solution (Levulan, DUSA) is applied to the entire affected area on face, torso and extremities for about 1 hour. The solution is removed from the skin surface. The areas are treated with contiguous, minimally overlapping pulses from a pulsed dye laser operating at 595 nm, with an irradiated spot size of about 10 mm in diameter, pulse duration of about 1.5 milliseconds, and fluence of about 5.0 J/cm². The procedure may be repeated at 2-to-6 week intervals until the lesions are eradicated.

### Example 4

This example provides one approach using a topically applied pro-photosensitizer for treating comedonal acne in a human.

Topical 20% 5-ALA solution (Levulan, DUSA) is applied to the entire affected area on face, torso and extremities for about 10 minutes. The solution is removed from the skin surface. The areas are treated with contiguous, minimally overlapping pulses from a pulsed dye laser operating at 585 nm, with an irradiated spot size of about 5 mm to about 7 mm in diameter, pulse duration of about 350 microseconds to about 550 microseconds, and fluence of about 2.0 J/cm², The procedure may be repeated at 2-to-6 week intervals until the lesions are eradicated.

### Example 5

100681 This example provides one approach using a topically applied pro-photosensitizer for treating basal cell carcinoma (BCC) in a human.

The BCC is prepared for photodynamic therapy by curettage. An oil-in-water emulsion is prepared by mixing 5-aminolevulinic acid (ALA) with GLAXAL™ Base (Shire Canada, Inc., Ontario) to produce a final concentration of about 20% (wt/wt) ALA. The emulsion then is applied to the basal cell carcinoma to give a dosage of about 25 mg/cm². The treated lesion then is covered with a light-blocking occlusive dressing for about 3 to about 24 hours.

The dressing then is removed and excess emulsion removed from the lesion surface. The lesion then is treated with contiguous, minimally overlapping pulses from a pulsed dye laser operating at 595 nm, with an irradiated spot size of about 10 mm in diameter, pulse duration of about 10 milliseconds, and fluence of about 8.0 J/cm². The procedure can be repeated at 2 to 6 week intervals, or until the basal cell carcinoma is eradicated.

### Example 6

This example provides an approach in which a photosensitizer is applied to an actinic keratosis.

A chloroform solution of egg phosphatidylcholine (Avanti Polar Lipids, Inc., Alabaster, Ala.) is mixed with hypericin (Sigma Chemical Co., St. Louis, Mo.) dissolved in methanol, and the mixture dried to a thin film using a stream of purified nitrogen gas. Traces of solvent then are removed by vacuum at room temperature for about 2 hours. Sterile isotonic saline previously purged with nitrogen is added to the lipid/hypericin mixture, and the resulting mixture shaken to form a homogenous suspension. The mixture then is allowed to stand for about 2 hours for further hydration. During preparation of the liposomal formulation of hypericin, exposure of the formulation to light is minimized. The resulting liposome composition comprises as a molar ratio of about 95.4 egg phosophatidylcholine: 4.6 hypericin.

The liposomal formulation then is applied to the actinic keratosis to form a layer covering the lesion and a surrounding margin of normal-appearing skin, and covered with a light-protective occlusive dressing. After a period of 1 to 6 hours, the dressing and remaining liposomal formulation is removed from the plaque. The lesion then is treated with contiguous, minimally overlapping pulses from a pulsed dye laser operating at 595 nm, with an irradiated spot size of about 10 mm, pulse duration of about 1.5 milliseconds and fluence of about 4.0 J/cm². The procedure may then be repeated at 2 to 6 week intervals, or until the actinic keratosis is eradicated.

### Example 7

This example provides another approach in which a photosensitizer is applied to an actinic keratosis.

Topical methyl ester ALA (Metvix, Photocure ASA, Norway) is applied to the actinic keratosis. Four hours later the dressing and excess methyl ester ALA is removed. The lesion is then treated with contiguous, minimally overlapping pulses from a pulsed dye laser operating at 595 nm, with an irradiated spot size of about 10 mm, pulse duration of about 1.5 milliseconds and fluence of about 4.0 J/cm², or below the purpura threshold. The patient is advised to protect the treatment site from sun exposure for 48 hours after treatment.

### Example 8

A patient with nevoid basal cell carcinoma syndrome is administered test site irradiation with a 585 nm pulsed dye laser with spot size of about 7 mm and pulse duration of about 10 ms. Test sites are located on the volar aspect of the forearm. The fluence is set to about 3.0 J/cm² and increased at about 0.5 J/cm² intervals until the purpura threshold is found. The purpura threshold is the minimum fluence that produces purpura within the full area of irradiation within 10 min after irradiation.

The patient is then administered the photosensitizer benzoporphyrin derivative monoacid (BPD-MA, verteporfin, QLT Phototherapeutics, Vancouver) at a dosage of about 8 mg/m² body surface area, intravenously. About 30 min to 3 hrs later, the individual BCC identified on the patient are irradiated with the pulsed dye laser, at a fluence of about 1.0 J/cm² below the purpura threshold previously determined. Each BCC is treated with contiguous, minimally overlapping pulses.

### Example 9

Topical methyl ester ALA (Metvix, Photocure ASA, Norway) to affected areas of facial skin in a patient with acne vulgaris, and covered with a light protective occlusive dressing.

About 45 minutes later the dressing and excess methyl ester ALA is removed. The lesion is then treated with contiguous, minimally overlapping pulses from a pulsed dye laser operating at 595 nm, with an irradiated spot size of about 10 mm, pulse duration of about 1.5 milliseconds and fluence of about 3.0 J/cm². The patient is advised to protect the treatment site from sun exposure for 48 hours after treatment.

### Example 10

Topical methyl ester ALA (Metvix, Photocure ASA, Norway) to affected areas of facial skin in a patient with acne vulgaris and acne scars, and covered with a light protective occlusive dressing. One to four hours later the dressing and excess methyl ester ALA is removed. The lesion is then treated with contiguous, minimally overlapping pulses from a pulsed dye laser operating at 635 nm, with an irradiated spot size of about 10 mm, pulse duration of about 1.5 milliseconds and fluence of 12 J/cm². The patient is advised to protect the treatment site from sun exposure for 48 hours after treatment.

### Example 11

A 20% ALA in an alcohol solution (Levulan, DUSA, USA) is applied to photoaged facial skin in a patient. About 30 min later the excess ALA on the skin surface is removed. The face is then treated with contiguous, minimally overlapping pulses from a flashlamp operating at a fluence of between about 20 and 50 J/cm², using a cut-off filter of 550 or 570 nm (Vasculight, Lumenis, Israel). The flashlamp produces a train of 2 or 3 micropulses that are between about 1 millisecond and 10 milliseconds in duration, separated by delays of 10 to 60 milliseconds. The patient is advised to protect the treatment site from sun exposure for 48 hours after treatment.

### Example 12

A 20% ALA solution in an alcohol (Levulan, DUSA, USA) is applied to photoaged facial skin in a patient, and covered with a light protective occlusive dressing. About 30 min later the dressing and excess ALA on the skin surface removed. The face is then treated with contiguous, minimally overlapping pulses from a flashlamp operating at a fluence of between about 40 and 70 J/cm², using a cut-off filter of 600 nm. The flashlamp produces a train of 2 or 3 micropulses that are between about 1 millisecond and about 10 milliseconds in duration, separated by delays of about 10 to 60 milliseconds. The patient is advised to protect the treatment site from sun exposure for 48 hours after treatment.

### Example 13

A 20% ALA solution in an alcohol (Levulan, DUSA, USA) is applied to photoaged facial skin in a patient, and covered with a light protective occlusive dressing. About 30 min later the dressing and excess ALA on the skin surface are removed. The face is then treated with contiguous, minimally overlapping pulses from a flashlamp operating at a fluence of between about 15 J/cm² and about 23 J/cm², with output from about 500 nm to about 650 nm, and about 20 milliseconds in pulse duration (Estelux with LuxG handpiece, Palomar, USA). The patient is advised to protect the treatment site from sun exposure for 48 hours after treatment.

### Example 14

Topical methyl ester ALA (Metvix, Photocure ASA, Norway) to affected areas of facial skin in a patient with rosacea, and covered with a light protective occlusive dressing. One to four hours later the dressing and excess methyl ester ALA is removed. The lesion is then treated with contiguous, minimally overlapping pulses from a pulsed dye laser operating at 595 nm, with an irradiated spot size of about 10 mm, pulse duration of about 10 milliseconds and fluence of about 7.0 J/cm². Dynamic cooling of the epidermis is used during laser treatment. The patient is advised to protect the treatment site from sun exposure for about 48 hours after treatment.

### Example 15

Olive oil (7.7 g, Sigma) and 8 g of a surfactant were mixed by shaking by hand at room temperature. Hypericin (15 mg, Sigma) was separately added to 1 g distilled water, then hand shaken with the lipid mixture. The resulting emulsion is applied to the facial skin of a patient with acne vulgaris, and covered with a light protective occlusive dressing. After one to four hours the dressing and excess hypericin emulsion is removed from the skin. The face is then treated with contiguous, minimally overlapping pulses from a flashlamp operating at a fluence of between about 30 and about 50 J/cm², using a cut-off filter of 570 μm. The flashlamp produces a train of 2 or 3 micropulses that are between about 1 millisecond and about 10 milliseconds in duration, separated by delays of about 10 to about 60 milliseconds. The patient is advised to protect the treatment site from sun exposure for about 48 hours after treatment.

### Example 16

Topical methyl ester ALA (Metvix, Photocure ASA, Norway) is applied to areas of normal skin with unwanted light-colored hair, and covered with a light protective occlusive dressing. About four to 18 hours later the dressing and excess methyl ester ALA is removed. The skin is then treated with contiguous, minimally overlapping pulses from a pulsed dye laser operating at 595 nm with an irradiated spot size of about 10 mm, pulse duration of about 6 milliseconds, at a fluence of about 10 J/cm² or just below the purpura threshold. Dynamic cooling of the epidermis is used during laser treatment. The patient is advised to protect the treatment site from sun exposure for about 48 hours after treatment. The procedure is repeated as necessary every 2 to 6 weeks until all unwanted hair is eradicated.

### Example 17

Topical 5-ALA (Levulan, DUSA) is applied to areas of unwanted blonde, gray or light-colored hair growth. About 3 hours later the excess ALA is removed. The skin is then treated with contiguous, minimally overlapping pulses from a system delivering light energy at between about 580 nm to about 980 nm and a fluence range of about 18 J/cm² to about 26 J/cm² of optical energy and radiofrequency energy of about 10 J/cm² to about 20 J/cm² (Aurora SR, Syneron). Coupling gel is used during laser treatment. Two passes are delivered. The patient is advised to protect the treatment site from sun exposure for about 48 hours after treatment. The procedure is repeated as necessary every two weeks until the unwanted hairs are eradicated.

### Example 18

Topical 5-ALA (Levulan, DUSA) is applied to areas of sebaceous hyperplasia. About 1 hour later the excess ALA is removed. The skin is then treated with contiguous, minimally overlapping pulses from a pulsed dye laser operating at 595 nm with an irradiated spot size of about 10 mm, pulse duration of about 10 milliseconds, at a fluence of about 10 J/cm² or just below the purpura threshold. Dynamic cooling of the epidermis is used during laser treatment. The procedure is repeated as necessary every month until the lesions are eradicated.

### Example 19

Topical 5-ALA (Levulan, DUSA) is applied to areas of oily skin. About 15 minutes later the excess ALA is removed. The skin is then treated with contiguous, minimally overlapping pulses from a pulsed dye laser operating at 595 nm with an irradiated spot size of about 10 mm, pulse duration of about 3 milliseconds, at a fluence of about 4.0 J/cm² or just below the purpura threshold. Dynamic cooling of the epidermis is used during laser treatment. The procedure is repeated as necessary every several months.

Each of the patent documents disclosed hereinabove is incorporated herein by reference in the entirety.

### Equivalents

The invention may be embodied in other specific forms without departing form the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

What is claimed is:

1. A method for treating a neoplastic or non-neoplastic dermatologic condition, the method comprising:
administering a photosensitizer or a pro-photosensitizer to a section of the skin affected with a neoplastic or non-neoplastic condition;
allowing the photosensitizer or pro-photosensitizer to accumulate in the skin tissue affected with the neoplastic or non-neoplastic condition; and
irradiating the section of the skin with a beam of light having a wavelength between about 500 nm and about 650 nm, a fluence rate of between about 100 W/cm² and about 40 MW/cm², and a fluence of less than about 60 J/cm², thereby causing a therapeutic injury to the section of skin affected by the neoplastic or non-neoplastic condition without causing purpura of the skin.

2. The method of claim 1 wherein what is administered is a photosensitizer.

3. The method of claim 1 wherein what is administered is a pro-photosensitizer and wherein the pro-photosensitizer is allowed to metabolize in the skin tissue affected with the neoplastic or non-neoplastic condition.

4. The method of claim 2 wherein the photosensitizer comprises at least one of a chlorin, a cyanine, a purpurin, and a porphyrin.

5. The method of claim 4 wherein the porphyrin is benzoporphyrin derivative monoacid.

6. The method of claim 2 where the photosensitizer comprises at least one of a bacteriochlorin, a bacteriocyanine, a bacteriopurpurin, and a bacterioporphyrin.

7. The method of claim 2 wherein the photosensitizer comprises at least one of a xanthenes and hypericin.

8. The method of claim 1 wherein the pro-photosensitizer comprises ALA.

9. The method of claim 8 wherein the pro-photosensitizer comprises an ALA derivative.

10. The method of claim 8 wherein the ALA derivative is an ALA ester.

11. The method of claim 8 wherein the ALA ester is ALA-methyl ester, ALA-n-pentyl ester, ALA-n-octyl ester, R,S-ALA-2-(hydroxymethyl)tetrahydropyranyl ester, N-acetyl-ALA, or N-acetyl-ALA-ethyl ester.

12. The method of claim 1 wherein the wavelength is between about 560 nm to about 600 nm.

13. The method of claim 1 wherein the wavelength is between about 600 nm and 650 nm.

14. The method of claim 1 wherein the beam of light is coherent.

15. The method of claim 1 wherein the beam of light is incoherent.

16. The method of claim 1 wherein the beam of light is continuous wave.

17. The method of claim 1 wherein the beam of light is pulsed.

18. The method of claim 1 wherein the fluence is less than about 30 J/cm².

19. The method of claim 1 wherein the fluence is less than about 20 J/cm².

20. The method of claim 1 wherein the fluence is less than about 12.0 J/cm².

21. The method of claim 1 wherein the fluence is less than about 7.5 J/cm².

22. The method of claim 17 wherein the a pulse duration is between about 1 microsecond and about 200 milliseconds.

23. The method of claim 22 wherein the pulse duration is between about 10 microsecond and about 10 milliseconds.

24. The method of claim 1 wherein the beam of light has a fluence rate of between about about 500 W/cm² and about 20 MW/cm².

25. The method of claim 1 wherein allowing the photosensitizer or pro-photosensitizer to accumulate and metabolite in the skin tissue comprises waiting for between about 0.1 to about 48 hours post-administration of the photosensitizer or pro-photosensitizer before irradiation.

26. The method of claim 1 wherein the neoplastic dermatological condition comprises at least one of actinic keratosis, skin cancer, Bowen's disease, and dysplasia,

27. The method of claim 1 wherein the non-neoplastic dermatological condition comprises at least one of verrucae vulgaris, acne vulgaris, acne conglobata, acne comedonica, papularpustular acne, acne inversa, acne fulminans, back acne, acne mechanical rosacea, sebaceous hyperplasia, oily skin, lichen planus, psoriasis, and eczema.

28. The method of claim 1 where the non-neoplastic dermatologic condition comprises at least one of unwanted hair, photoaged skin, and wrinkles.

29. The method of claim 1 wherein the photosensitizer or pro-photosensitizer is administered orally, topically, or parenterally.

30. The method of claim 1 wherein the photosensitizer or pro-photosensitizer is administered as a component of a formulation comprising a pharmaceutically acceptable carrier or excipient.

31. The method of claim 1 wherein the therapeutic injury results in the reduction of at least one of the surface area, the depth, and the amount of the skin affected by the neoplastic or non-neoplastic condition.

32. The method of claim 1 further comprising heating the section of the skin with a pulse of radio frequency before, during, or after the irradiation of the section of the skin with a beam of light.

33. A method for treating a neoplastic or non-neoplastic dermatologic condition, the method comprising:
applying a pro-photosensitizer to a section of the skin affected with a neoplastic or non-neoplastic condition;
allowing the pro-photosensitizer to accumulate and metabolize in the skin tissue affected with the neoplastic or non-neoplastic condition; and
irradiating the section of the skin with a pulsed laser beam having a wavelength between about 500 nm and 650 nm, a fluence less than about 20 J/cm², and a pulse duration between about 10 microseconds and 40 milliseconds, thereby causing a therapeutic injury to the section of skin affected by the neoplastic or non-neoplastic condition without causing purpura of the skin.
